(19) United States
(12) Patent Application Publication
(10) Pub. No.: US 2014/0275256 A1
(43) Pub. Date: Sep. 18, 2014

(54) Transepithelial Methods Of Using Gamma Aminobutyric Acid Compositions For Pain Relief

(71) Applicants: Michael H. Moskowitz, Mill Valley, CA (US); MarIa DePolo Golden, Jacksonville, FL (US); Dana Gordon, Pebble Beach, CA (US)

(72) Inventors: Michael H. Moskowitz, Mill Valley, CA (US); MarIa DePolo Golden, Jacksonville, FL (US); Dana Gordon, Pebble Beach, CA (US)

(21) Appl. No.: 14/120,463

(22) Filed: May 21, 2014

Related U.S. Application Data
(62) Division of application No. 13/067,827, filed on Jun. 29, 2011.
Application No. 13/067,827 is a continuation-in-part of application No. 12/692,935, filed on Jan. 25, 2010, now abandoned.

(51) Int. Cl. A61K 9/00; A61K 31/197

(52) U.S. Cl. CPC A61K 9/0014; A61K 31/197
USPC 514/561

(57) ABSTRACT

Disclosed are methods for alleviating, relieving, inhibiting, and eliminating acute or chronic pain in a patient suffering from such pain by contacting epithelial tissue of the patient with exogenous GABA (gamma-amino butyric acid).

The epithelial tissue involves skin. The GABA is provided in compositions such as water, saline, buffered saline, lotions, semi-solids such as, gels, creams, pastes, salves, ointments, solids such as impregnated patches, impregnated rub-on solid compositions from which GABA may be applied onto the skin.

### CROSS REFERENCE TO RELATED APPLICATIONS

This application is a divisional application of U.S. Ser. No. 13/067,827 filed Jun. 29, 2011 which is a continuation-in-part of U.S. Ser. No. 12/692,935 entitled Transdermal Application of Gamma Amino Butyric Acid For Pain Relief, filed Jan. 25, 2010.

### STATEMENT REGARDING FEDERALLY SPONSORED RESEARCH OR DEVELOPMENT

(Not Applicable)

### STATEMENT REGARDING FEDERALLY SPONSORED RESEARCH OR DEVELOPMENT

### REFERENCE TO A SEQUENCE LISTING, A TABLE, OR A COMPUTER

PROGRAM LISTING APPENDIX SUBMITTED ON COMPACT DISC (SEE 37 CFR 1.52 (e)(5))

(Not Applicable)

### BACKGROUND OF THE INVENTION

A. Field of the Invention

This invention relates to the relief or inhibition of chronic or acute pain by administering to a subject suffering from pain, a therapeutically effective amount of exogenous gamma aminobutyric acid (GABA) by contacting appropriate epithelial tissue of the subject.

More particularly, it relates to the application of formulations of GABA to epithelial tissue, primarily skin, nasal mucosa or oral mucosa, of a patient suffering from pain, to introduce pain-relieving amounts of exogenous GABA into the patient's body.

More particularly still, it relates to the application of exogenous GABA to the local site of a pain source of a subject to inhibit or relieve local pain at the affected site.

B. Prior Art

GABA is a naturally occurring polypeptide neurotransmitter present in the human body and also produced by tissue in response to an injury or other insult. It is utilized in the central and peripheral nervous systems. It is the chief inhibitory neurotransmitter in the central nervous system and the peripheral nervous system of the human and is known to modulate pain in the biochemistry of the human. It functions in balance with glutamate to alter the occurrence of pain and inhibition of pain depending on the amount of GABA released in response to the pain. GABA that occurs naturally in the body is referred to herein as endogenous GABA. GABA that is used in the invention is variously referred to as exogenous GABA, or simply as GABA when the context is clear that exogenous GABA is intended or apparent.

GABA and glutamate are the two main neurotransmitters in the brain at the base of balancing brain activity. Together these molecules account for 90% of all of the neurotransmitters in the brain. Simply put, glutamate turns on nerves and GABA turns them off. Generally, all chronic disease activity, including pain, in the brain involves the excitatory glutamate overwhelming the inhibitory GABA. GABA acts like a brake on glutamate to prevent excitation (and therefore, pain) from running away with the brain and communicates with the body to release its own excitatory (inhibiting) chemicals.

In the brain pathways responsible for controlling pain, GABA is often outmatched and glutamate dominates in persistent pain states. As interactions between other processes in the brain shift toward this state of alert, more of the main pain neurotransmitter is released, leading to a cascade of networks of pain-processing cells. As pain processing grows there is an increase in the firing of nerves in the brain's pain networks, adding to the release of more inflammatory chemicals and hence more pain.

The body would normally respond in a case such as that just described, by releasing endogenous GABA from the body tissue involved in the injury. The GABA would act as a brake on excessively firing peripheral nerves and also work directly on inflammatory immune system cells to shut off the inflammation. In chronic pain subjects, the amount of GABA in the peripheral nervous system is not enough to shut off these nerves and the input persists.

The pain-suffering patient becomes increasingly helpless and feels that the only option is to suppress the pain. This is usually done by taking undesirable CNS suppressing pain killers that are only partially successful, leaving the patient to suffer with both the pain and the adverse CNS effects.

Exogenous GABA has been widely used in various forms and for various reasons. The compound is widely available over-the-counter in numerous forms including food products, such as rice and chocolate; drinks such as teas, coffees and sodas; oral supplements such as pills, tablets, powders and various liquid formulations; and topical versions such as creams, lotions, gels and the like.

The prior art variously describes exogenous GABA as being useful in a wide range of conditions and maladies, among which are: treatment of depression, sleep pattern disorders, anxieties, neuroses, hypertension, loss of libido, digestive disease and the like.

Despite the attribution of many properties to GABA in the prior art, some of which may or may not be factual, the prior art reports of various formulations containing GABA, various indications and the various routes of administration to patients, as will be discussed further hereinbelow, have not included any reports of the administration of exogenous GABA as set forth in the invention, to subjects suffering from pain for the purpose of reducing, inhibiting or eliminating the pain in such subjects.

### BRIEF SUMMARY OF THE INVENTION

The treatment of chronic or acute pain takes many forms and can be a lifelong battle using many different types of modalities. Some of these treatment modalities include injections, oral medications, patches and creams.

Although the treatment for the control of pain, particularly chronic pain, is necessary for the comfort of the patient, it also may carry with it some undesirable side effects. One of the common side effects is the impaired judgment that is caused by the pain medicine because most, if not all pain medicine, affects the person's brain. This side effect may produce impaired judgment, delayed reaction time, cognitive deficits, dependency, hallucinations, grogginess, and addiction among others.

In order for endogenous GABA to function in the body and act to relieve pain, properly functioning receptors are needed. In the person who is suffering from chronic pain, the endogenous GABA is overwhelmed as a result of spontaneous, persistent and excessive firing of the injured peripheral nerves. If there is not enough GABA to cover the receptors, the nerves that are affected continue to send signals to the brain and the pain level of the person increases and persists. The present invention surprisingly presents methods which deliver exogenous GABA to turn on the GABA receptors in the formerly continuously firing nerves to yield the pain relief described herein.

This invention thus comprises a method for alleviating pain in a subject suffering from pain, and in many cases eliminates pain for long periods of time, which comprises administering to the said subject suffering from pain, a therapeutically effective amount of exogenous GABA, to epithelial tissue, primarily to skin.

The preferred application site is the local site of a pain source of a subject when it is desired to inhibit or relieve the local pain at the affected site. The preferred application site when it is desired to inhibit or relieve the pain of a subject systemically can also be delivered locally to the skin

The GABA may be supplied in any convenient formulation. The compositions described in this specification in terms of physical forms thereof may range from freely flowing liquids such as water and other liquids of similar viscosity such as saline, to more viscous liquids such as flowable lotions, to semi-solids which themselves may or may not be somewhat flowable but are layerable as a film or thin layer, such as creams, gels, pastes, salves, ointments, impregnated adhesive patches and the like, some of which may be of amorphous character or have no particular shape except that of their container, to solid forms of absorbable compositions applicable as a film or layer having the consistency of, for example, lipstick, lipbalms, rub-on deodorants and the like and topical impregnated adhesive patches containing absorbable GABA formulations.

The methods of the invention are practiced by bringing the appropriate GABA-containing formulation into contact with the epithelial tissue of the selected route of administration for a period of time sufficient to allow the GABA to be absorbed by the tissue.

The invention is thus directed to the discovery of a new use, i.e., that of pain alleviation of the known material, GABA.

### DETAILED DESCRIPTION OF THE INVENTION

To our knowledge, no one has described the actual use of GABA as a compound which can very quickly inhibit and ameliorate and eliminate pain and in particular chronic pain, often for long periods of time, from a variety of sources in an affected subject.

Pain reduction has been achieved herein through the use of a formulation comprising GABA which is easily topically applied to the skin at a local site of the pain source.

The invention thus encompasses in one embodiment, a method of using exogenous GABA to inhibit the pain of a subject affected with said pain which comprises topically applying a therapeutically effective amount of exogenous GABA to the local site of the pain source. As a result of the methods of the inventions, pain relief has been obtained by subjects ranging from significant and substantial reduction in pain to complete relief of pain for prolonged periods of time.

The GABA is preferably applied as a topical skin composition such as a viscous liquid such as a lotion, or semi-solid cream, paste, salve, gel or similar cohesive form, or a solid form, which can be conveniently rubbed into, and adhere to, the skin, or a solid impregnated adhesive topical patch which adheres to the local site and from which GABA may be absorbed into the skin.

The topical application to skin is preferred for episodes of local pain origin such as muscle cramps, pain due to injury or exercise where pain is associated with a local causative source, and in particular chronic pain.

The utility and success of the invention may be achieved through the use of an almost boundless array of formulations. Various formulations for topically applied skin formulations or compositions as vehicles for the GABA, such as solids, semi-solids, and liquid vehicles, abound. Any of these may be employed provided they are not inhospitable to, or incompatible with, the GABA component.

The formulation may also contain active ingredients other than GABA although this is not necessarily preferred. Nor are any additional active ingredients necessary in the ability of GABA to relieve pain according to the invention. We have discovered that exogenous GABA alone is quite efficacious in relieving pain. Of course, the normal and usual excipients, moisturizers, adjuvants, preservatives and the like used in the art, may be employed as desired. These materials are too numerous to mention exhaustively, but the selection of specific ingredients is well within the skill of the art. Presented in the examples below are representative ingredients which have been sued in preparing compositions used in the invention. Merely as illustrative, any of the following materials alone or in various combinations are used in the art and may be used in the present invention. purified water, phospholipids, isopropyl myristate, cetearyl alcohol, stearyl alcohol, cetyl alcohol, caprylic/capric triglyceride, ceteareth-20, wheat germ oil, glycerin, dimethicone, magnesium aluminum silicate, xanthan gum, polyacrylamide, C13-C14 isoparaffin, laureth-7, disodium EDTA, BHT (butylated hydroxytoluene), phenoxyethanol, methylisothiazolinone, methylchloroisothiazolinone, soya lecithin, isopropyl palmitate, sodium hydroxide, EDTA (edetate disodium), steric acid, glyceryl monostearate, simethicone, urea, polyoxyl 40 stearate, methylparaben, propylparaben, imidurea and ethyl alcohol.

The invention in its simplest and most direct form is quite accessible and comprises a method of providing a therapeutically effective amount of GABA in the formulation of the user's choice. GABA dissolved in saline or even simply water, with or without a preservative, will be sufficient. In some cases, an oil base might be utilized in which event emulsifiers may be used as appropriate. In the topical skin form, GABA in a simple, skin formulation such as a cream, ointment, salve, paste or gel or any other semi-solid or solid vehicle as aforesaid, will serve quite well. The term “skin compositions” or “skin formulations”, or like terms as used herein are meant to apply to any such vehicle, irrespective of their specific non-GABA ingredients, whether included in the above list, or not.

The results of the invention are quite surprising and unexpected in view of the collected wisdom concerning accepted drug selection in the art. In this regard, it is generally thought that when bioactive materials, medicaments, drugs, and the like are sought, one looks to compounds that cross the blood-brain barrier in order to be systemically effective. GABA is known not to cross the blood-brain barrier, however, and therefore would not be expected to elicit a systemic response. The fact that it does create such an effect is of surprise in this invention, and the fact that the results in many cases last for a prolonged period of time after dosing is indeed unexpected. The art would have been motivated to avoid a compound of this type for the alleviation, inhibition or elimination of pain as described herein.

In conventional pain management, much of the medicine is absorbed into the bloodstream soon after being taken through the usual oral ingestion route. Because the conventional pain medicine enters the bloodstream, however, it will usually cross the blood-brain barrier because the molecules are designed to do so. Consequently, a major side effect of most pain medicines that are taken orally is the effect on the cognitive abilities of an individual because it has crossed the blood-brain barrier and impacted the functioning of the brain.

Although pain relief might be achieved, the patient nevertheless may suffer undesireable side effects, such as decreased immediate recall, decreased short term memory and decreased word finding abilities, as well as increased fatigue, drowsiness, confusion and memory disturbance. This is because once these medications cross the blood brain barrier, they are distributed throughout the whole brain, not just to the targeted sites.

In the present invention, the GABA formulation has a focused local effect when applied topically to the skin and because of the large size of the GABA molecule, is limited in its ability to cross the blood-brain barrier. Therefore, a principal advantage of the invention for the topical application route, is that pain-relief is obtained without loss of cognitive ability. Moreover, because the absorption is through the skin, the effect is often immediate in many cases and very rapid in others.

Another drawback to pain medicine which enters the blood stream is the length of time required for the medication to have the desired effect. Typically in orally supplied medicines for example, the medicine must be absorbed via blood vessels from the gastrointestinal tract, and eventually enters the bloodstream from that site. From the bloodstream, the medication must then go through another filtering process to cross the blood-brain barrier. There is thus, a significant delay between the ingestion of the pain medicine and the desired effect.

While we do not intend to be bound by any particular theory of the pharmacological effect of the invention, it appears that the brain, in some way, monitors the level of GABA present in the circulating blood-stream. When that concentration is high enough from the exogenously supplied GABA, the brain appears to send a signal throughout the body to turn off the excitatory nerves responsible for pain occurring anywhere in the body. The result is a systemic reduction, inhibition or even elimination of pain anywhere in the body where the nerves are firing, without regard to the cause or source. As has been noted above, the results are often apparent for prolonged periods of time even after dosing has been discontinued.

In local areas of topical skin administration to skin the exogenously added GABA tends to concentrate in the local areas where it overwhelms the pain-inducing effect of inflammation or nerve injury on the excitatory nerves, reducing the electrical signals of the local nerves. Thus, local relief from the source of pain at the local site is obtained without ever increasing substantially the concentration of GABA in the bloodstream or the brain.

In topical skin application of the present invention, instead of entering the bloodstream, the GABA affects the local peripheral nervous system and prevents transmission of pain signals to the brain by allowing the GABA receptors to function properly. As a result, the patient obtains pain relief without the unfortunate side effects usually encountered with conventional pain medications. It should be noted that GABA is not a topical anesthetic like lidocaine and the other “-caine” suffix compounds such as benzocaine, xylocaine and the like and does not exhibit its pain-relieving properties in that manner.

The GABA present in the skin compositions works on the peripheral nervous system as well as the skin, fascia and the underlying muscle. By increasing the amount of GABA by application of this formulation the receptors work on local nerve axons to inhibit electrical activity and the level of pain decreases. Additionally, endogenous GABA has demonstrated scientifically proven properties of blocking inflammation separate and independent of its effect as the body's main inhibitory neurotransmitter by working in the extracellular matrix. Application of exogenous GABA locally, increases the availability of GABA to decrease inflammation where it is applied.

The topical approach to a local site usually takes effect between 2-20 minutes after application.

### Therapeutically Effective Amounts of GABA

The compositions containing GABA for use in the present invention generally comprise a therapeutically effective amount of GABA when a reasonable volume of the composition is applied using the routes of administration described herein. Actual amounts used may vary according to the severity and type of pain.

A therapeutically effective amount of GABA when used in semi-solids such as ointments, gels, pastes, creams, salves and the like in topical skin applications, suitably include from about 100 to about 300 mg, preferably about 150 to about 250 mg GABA, per 10 square cm of applied surface. Clinically, we use about 200 mg GABA per 10 square cm of application site surface quite successfully from once per day to 4 -6 times per day as needed.

The effective dosage regimen can be administered as frequently as needed, depending upon the severity of the pain, from once per day to every 4-6 hours, daily as needed.

### Concentration of GABA

The concentration of GABA in the compositions applied suitably includes:

for liquids, an amount of GABA from about 5% to about 25% or more, GABA by weight and preferably from about 8% to about 12% GABA by weight of solution, (about 50 mg to about 250 mg GABA per ml, and about 80 mg to about 120 mg GABA per ml, respectively).

For semi-solids and other skin formulation type compositions such as pastes, creams, gels, salves, ointments and the like, the concentration of GABA suitably ranges from about 100 mg to about 300 mg per ml of composition, i.e. from about 10% to about 30% by weight per ml, and preferably from about 150 mg to about 250 mg GABA per ml of composition, i.e., from about 15% to about 25% by weight per ml. The compositions are effectively rubbed into the affected site for about 2 to 3 minutes or so and allowed to air dry.

As can be seen from the foregoing, the methods of the invention address the major disadvantages of pain medication which involves absorption into the central nervous system. With this invention there is rapid relief with virtually no impact on cognitive function or other CNS generated side effects.

Pain relief is virtually immediate and does not require the processing time for oral medications. The invention is useful for acute pain as well as for chronic pain.

Persons with chronic pain who have been treated with the methods of the invention have experienced immediate long term pain relief with little to no adverse side effects. Many of the patients using this medication for a period over months have reported greatly improved quality of life and reduced or eliminated dependence on oral pain medicines.

Typically, patients usually follow a regimen of application every 4-6 hours as needed. No significant adverse effects have been observed upon prolonged use of several months or more.

Over the course of developing the methods of the present invention, we have used topical formulations in the form of creams of various compositions in tests on a large group of patients with persistent pain from various causes. In the vast majority of cases, the relief was immediate and substantial. The reduction in standard subjective pain scales in ninety-two (92) patients showed greater than fifty (50) percent reduction of pain within twenty minutes on 82.61% of the patients and 100% pain relief in 45.65% of the patients. These patients experienced far more rapid and profound pain relief than any other oral, topical or transdermal medication and none reported any significant side effects.

### EXAMPLES

A. The following patients having the starting pain level noted, were administered the topical composition containing GABA described in paragraph [0073]. Pain levels were expressed at 2, 5 and 20 minutes after dosing as shown below with the Results indicated.

| Patient | Start Pain | Two | Five | Twenty | | Percent |
|---|---|---|---|---|---|---|
| (Age) | Score | Minutes | Minutes | Minutes | Change | Change |
| DA (58) | 10 | 2 | 0 | 0 | 10 | 100.00 |
| NM (59) | 6 | 3 | 1 | 0 | 6 | 100.00 |
| TS (62) | 9 | | 0 | 0 | 9 | 100.00 |
| PB (52) | 8 | 2 | 0 | 0 | 8 | 100.00 |
| AM (34) | 8 | 0 | | 0 | 8 | 100.00 |
| FT (63) | 5 | 0 | | 0 | 5 | 100.00 |
| DS (43) | 4.5 | 0 | | 0 | 4.5 | 100.00 |
| JR (82) | 6 | 3 | 0 | 0 | 6 | 100.00 |

### Results

Long Term Response

DA—reduces chronic low back and leg pain level to 0/10 Consistently—reduced opioids

NM—reduces chronic neck and low backpain level to 0/10 Consistently—reduced opioids

TS—keeps Neuropathic pain and Complex Regional Pain Syndrome

Pain manageable and reduces it to 2/10—reduced opioids

PB—lowers fibromyalgia pain, chronic degenerative neck pain and chronic degenerative back injury pain by half consistently—reduced opioids

AM—reduced overall chronic post-infectious neuropathic pain level consistently, helps with flares

FT—Average chronic degenerative back and neuropathic pain level dropped from a 6.5 to 3 in a year, no injections for pain in a year

DS—reduced degenerative thoracic and lumbar and neuropathic pain from an average of 6 to an average of 1.5, more active, reduced opioids

JR—Reduced her chronic back pain from 6 to 0 consistently

The above results dramatically demonstrate the alleviation and elimination of pain using the invention.

The topical cream formulation was prepared from the following:
GABA—20% by weight per ml of final composition.
CREAM BASE—80% by weight per ml of equal weights of LIPODERM® and VAN PEN®, two skin formulations available commercially from PCCA Corp. of Houston, Tex. and containing primarily the ingredients selected from those set forth in paragraph [0031] hereof
The GABA is dispersed in ethylene glycol to form a thickened liquid which is homogeneously blended with the CREAM BASE to form the topical cream formulation.

The amount of cream used on each patient was one ml per 10 square cm of applied skin surface. The ratio of LIPODERM® to VAN PEN® may be adjusted to provide varying cream consistencies.

What is claimed is:

1. The method for systemically alleviating pain in a subject suffering from pain which comprises administering to said subject a therapeutically effective amount of exogenous GABA by contacting skin of the subject with a composition comprising GABA and allowing a therapeutically effective amount of GABA to be absorbed by the skin.

2. The method of claim 1, wherein the subject is human.

3. The method of claim 2, wherein the GABA is present in a topical skin composition.

4. The method of claim 3 wherein the topical skin composition comprising GABA has a consistency selected from the group consisting of water, free-flowing liquids of a viscosity higher than water, semi-solids and solids which can be applied to the skin.

5. The method of claim 4, wherein the amount of GABA applied per treatment ranges from about 100 mg to about 300 mg of GABA per 10 square cm of application site.

6. The method of claim 4, wherein the topical composition comprises from about 10% to about 30% of GABA based on the weight of the total composition.

7. The method of claim 4, wherein the topical skin composition is selected from the group consisting of creams, gels, ointments, pastes, salves and lotions.

8. The method of claim 5, wherein the GABA is applied to a patient in a single or multiple daily dosage regimen.

9. The method of claim 8, wherein the composition is administered daily and the daily dose is from about 150 mg to about 1200 mg of the GABA per 10 square cm of application site.

10. The method of claim 9, wherein the composition is administered from 1 to 4 times daily.

11. The method of claim 3, wherein the GABA composition is applied to the skin at the local source of the subject's pain.

12. The method of claim 11, where the subject is suffering from chronic pain.

13. The method of claim 11, wherein the subject is suffering from acute pain.

14. The method of claim 5, wherein the amount of GABA applied per treatment ranges from about 150 mg. to about 250 mg of GABA per 10 square cm of application site.

15. The method of claim 4 wherein the topical composition comprising GABA has a consistency of water or a free-flowing liquid having a viscosity higher than water and the GABA comprises about 5% to about 25% by weight of the total composition.
